(19) United States
(12) Patent Application Publication
(10) Pub. No.: US 2004/0048252 A1
(43) Pub. Date: Mar. 11, 2004

(54) Method and Device For the Synthesis and the Analysis of Suppert-Bound Arrays of Oligomers, Especially of Primer Pairs for PCR, as well as Oligomer-Carrying Supports

(76) Inventors: Frank Breitling, Heidelberg (DE); Annemarie Poustka, Heidelberg (DE); Karl-Heinz Gross, Dossenheim (DE); Frieder Breitling, Wiesloch (DE)

Correspondence Address: INTELLECTUAL PROPERTY / TECHNOLOGY LAW, PO BOX 14329, RESEARCH TRIANGLE PARK, NC, 27709, US

(21) Appl. No.: 10/203,082

(22) PCT Filed: Feb. 2, 2001
(86) PCT No.: PCT/DE01/00435

(30) Foreign Application Priority Data
Feb. 3, 2000 (DE) 100 04 659.2-44
Jun. 21, 2000 (DE) 100 30 588.1

(51) Int. Cl. C12Q 1/68; C12M 1/34; B05D 3/00

(52) U.S. Cl. 435/6; 435/287.2; 427/2.11

(57) ABSTRACT

For synthesis of a support-bound array of oligomers with free ends A, whereby a temporary protecting group is provided on the 5′-OH end of the oligomers and permanent protecting groups on reactive side groups, it is proposed that the temporary protecting group is removed from the end B after combinatorial synthesis of the oligomers onto the support, the free ends B are then cross-linked even before splitting of the permanent protecting groups, then the covalent bond of the synthesised oligomers can be removed via the end A onto the support, resulting in free ends A, and a portion of the synthesised oligomers binds covalently to the support also after removal of the ends A from the support.

This invention relates to methods and devices for the synthesis and analysis of support-bound arrays of oligomers, especially of primer pairs for PCR (Polymerase Chain Reaction [Polymerase Kettenreaktion]), as well as supports with oligomers. In particular the invention relates to methods and devices for parallel synthesis of complex oligomer libraries, especially oligonucleotide libraries.

Here the term “oligomer” or “oligonucleotide library” designates all the many different, oligomers, peptides, nucleotides or ribonucleotides bound on a support at defined points, whereby the different oligomers or (ribo)nucleotides should be arranged as compactly as possible. Such molecule libraries originate especially from combinatorial synthesis of a limited number of monomers.

The term “complex” designates molecule libraries with more than 10² different agents, but in particular molecule libraries with more than 10⁴ different agents.

The term “PCR-array” designates all the many different oligonucleotide pairs, bound on a support at defined locations with a free 3′OH end. The characteristic feature of these primer pairs is to bind suitable DNA templates, by means of which an amplificate bound to the support according to primer pair in each case originates as defined by location under PCR conditions.

A range of methods for combinatorial synthesis of molecule libraries is already known, including printing processes for combinatorial synthesis, in which liquids are applied to a support as defined by location. The ink-jet printer (patent application WO 97 44134 A, Incyte Pharmaceutical Inc., 1997; or U.S. Pat. No. 5,449,754, Nishioka, 1995) is mentioned here as a prototype of these methods. Another method for combinatorial synthesis uses a compact disc as support (WO 98 12559 A, Demers, 1998). Yet another method employs a controllable chip as support (U.S. Pat. No. 5,667,667, Southern, 1996). And another method uses a colour laser printer with modified toner particles (PCT/DE99/03982). Lithographic methods are also known (U.S. Pat. No. 5,424,186, Very large scale immobilized polymer synthesis, Fodor et al., 1991), in which sequentially:

defined locations are irradiated with light

these locations are activated for covalent coupling of monomers for combinatorial synthesis

together with the non-activated locations these activated locations are then put into contact with a liquid solvent, in which suitable monomers are dissolved for combinatorial synthesis

which can then couple to the support according to location.

repetition of this procedure with the various monomers first results in localised coupling of a first layer of different monomers

further repetition of this procedure leads to localised coupling of other layers of different monomers and thus to localised structuring of oligomers.

In the same way there are methods known, in which oligonucleotides, in particular primer pairs for PCR, are attached to a support with the aim of obtaining a high-grade parallelisable PCR (U.S. Pat. No. 5,641,658, U.S. Pat. No. 6,060,288 and U.S. Pat. No. 6,090,592). The considerable advantage of these methods is that the primer pairs are coupled to the support covalently. The primers are also used to couple the PCR products to the support, so that no cross-contamination by primers or PCR products can arise. The result of this is that various PCR reactions must be separated from one another not by separate reaction chambers. This goal is achieved very elegantly solely by the spatially separate coupling of primer pairs to a support.

A combination of methods for the combinatorial synthesis of oligonucleotides using the abovedescribed methods of a high-grade parallelisable PCR is therefore offered, since the combinatorial synthesis of oligonucleotides is particularly suited to manufacturing large numbers of different oligonucleotides in a comparatively simple manner. The U.S. Pat. No. 6,156,494 also shows a way in which combinatorial synthesis can be performed, this time with the aim of obtaining arrays of primer pairs for the PCR. An array of glass fibres in this case serves as support.

In the abovementioned inventions little attention is given to the fact that oligonucleotides in particular, but also other types of oligomers, exhibit polarity. Oligonucleotides have e.g. a 5′OH end and a 3′OH end, while peptides have an amino and a carboxyl terminal end. This polarity also applies to each individual module of the oligomer, e.g. nucleotides in the case of oligonucleotides, or amino acids in the case of peptides. If the aim is for example the combinatorial synthesis of an array of primer pairs for a subsequent PCR, then the oligonucleotides bound on the support must have a free 3′OH end, because only then can they act as a substrate for a polymerase.

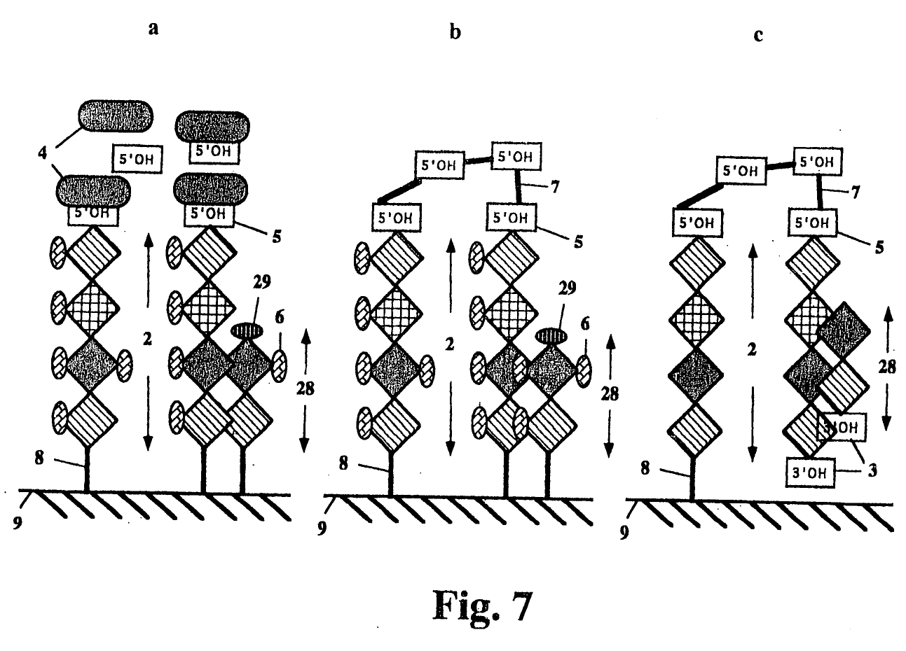

Little attention also is given to a particular property inherent in all chemical syntheses: no chemical synthesis runs completely finitely. Inter alia therefore only synthesis efficiencies of 90-96% yield are obtained in the individual synthesis steps with the synthesis of oligonucleotides or peptides (based on the monomer modules), as a rule. This fact is very well known to the expert, which is why a “capping” step is normally introduced to such syntheses. The majority of the free (i.e. not reacted) ends of the solid phase-coupled growing oligomer reacts with a reactive “capping” molecule (FIG. 7; 29), which is added in excess. The resulting effect is that this oligomer can no longer participate in further synthesis steps. With an increasing number of synthesis steps, i.e., the longer the oligomer becomes, the more synthesis artefacts coupled to the support there are, which after 30 synthesis cycles at the latest represent the overwhelming majority of the manufactured molecules.

The present invention offers an amazingly simple solution as to how these synthesis artefacts can be cleaned off in situ and therefore parallelised high-grade. The invention is therefore suited to in situ cleaning of the desired end products on a support, in particular with a complex array. This is in many cases the requisite for a meaningful use of arrays, as many observable effects are destroyed by contaminants. The simultaneous effect of the invention is that the oligomers anchored to the support are “reversed”, so that the end first anchored to the support, in particular the 3′OH end of an oligonucleotide, is then freely accessible for appropriate enzymatic activity. At the same time the invention offers a particularly simple way to manufacture PCR arrays (arrays of primer pairs). A considerable technical advantage here is that according to the present invention the especially accessible modules last coupled to the support are cross-linked. In this way steric problems inter alia are extensively avoided by the possibly very bulky side protecting groups. With increasing length these load the oligomers built on the support, in particular access to the “linker”, which attaches the oligomer to the support. It is especially a very well-known problem for the expert if the “linker” itself has to take on the deciding role with “reversing” of the synthesised oligomer, as is described e.g. in U.S. Pat. No. 5,550,215: “Polymer reversal on solid surfaces”.

This invention should therefore in particular enable synthesis of primer pairs with a free 3′-OH end, which are suited for subsequent PCR analysis and which result from combinatorial synthesis as a pair at each defined location on a support.

Most of the previously known solid phase synthesis methods for the synthesis of oligonucleotides and the combinatorial synthesis of oligonucleotide arrays anchor the growing oligonucleotide with the 3′-OH end on the support, whereas new monomers are coupled to the 5′-OH end after splitting of the temporary protecting group. On completion of synthesis all protecting groups are split off. In this last step the method of anchoring to the support decides whether the oligonucleotide remains with its 3′-OH end bound on the support, or whether it merges into the soluble fraction.

Monomers are used in an alternative method, where the temporary protecting group is situated at the 3′OH end. These monomers give rise to oligonucleotides with a free 3′OH end, which bind to the support with their 5′OH end. Monomers of this type for oligonucleotide synthesis are sold by the company Glensearch (www.glenres.com). To date this type of monomer has been employed inter alia for special applications such as more stable antisense oligonucleotides, or for the synthesis of double strands hybridising parallel to one another. One of the reasons for this is that these monomers are very expensive.

These monomers have so far not been used for the synthesis of an array of primer pairs. Yet it would be an advantage if defined primer pairs each with free 3′-OH ends could be fixed not only at a defined location, but also cleaned in situ at the same time, as e.g. thousands of PCR reactions can be performed in parallel very easily. The reason for this is that in the process the primers are not mixed thoroughly, but remain fixed to the support according to location.

The previous technical methods form an obstacle to this, as the synthesised oligonucleotides bind to the support mostly with their 3′OH end. At the same time there is no in situ accumulation of the synthesised oligomers.

Based on this the aim of the invention is to provide improved methods and devices for synthesis or analysis of oligomers on a support, in particular of primer pairs for PCR (Polymerase Chain Reaction [Polymerase Kettenreaktion]), as well as improved supports with oligomers cleaned in situ.

This task is solved by methods or devices having the characteristics of the independent claims. Advantageous realisations or embodiments are the subject of the sub-claims.

Further details and advantages of the invention will emerge from the following description of some examples in conjunction with the diagram, in which:

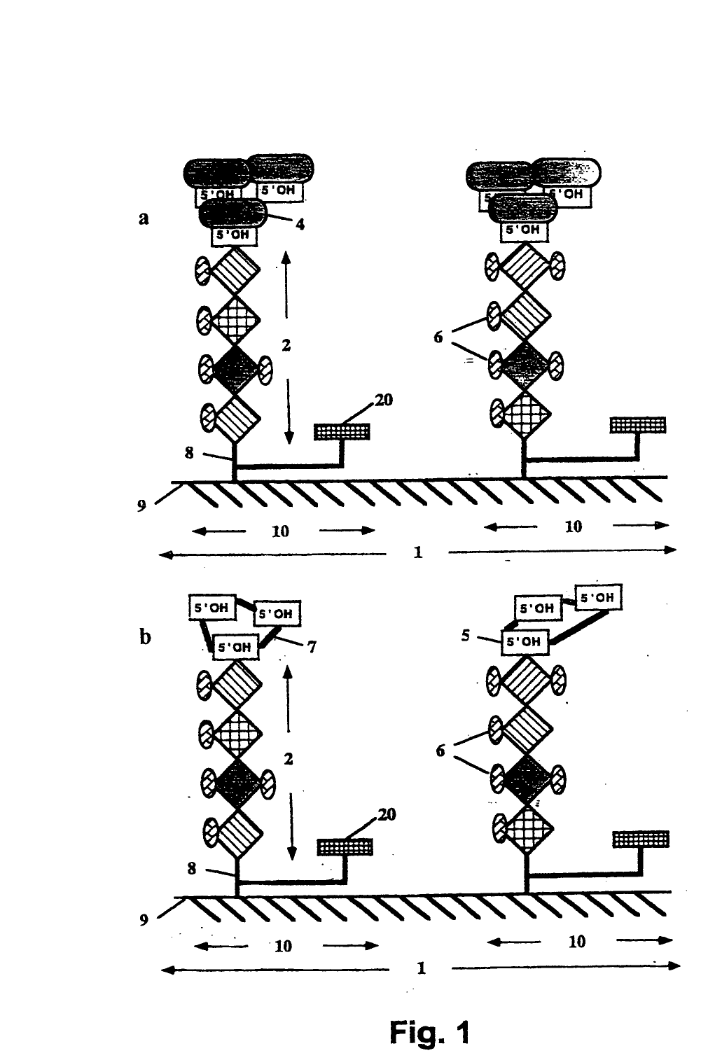

FIG. 1a diagrammatically illustrates the splitting of the temporary protecting groups 4 on the 5′-OH end 5 of the oligonucleotides 2 following combinatorial synthesis of a first array 1 of oligonucleotides 2 bound at specific locations 10 on the support 9,

FIG. 1b diagrammatically illustrates the formation of cross-linking 7 between the now free 5′-OH ends 5 of the oligonucleotides 2,

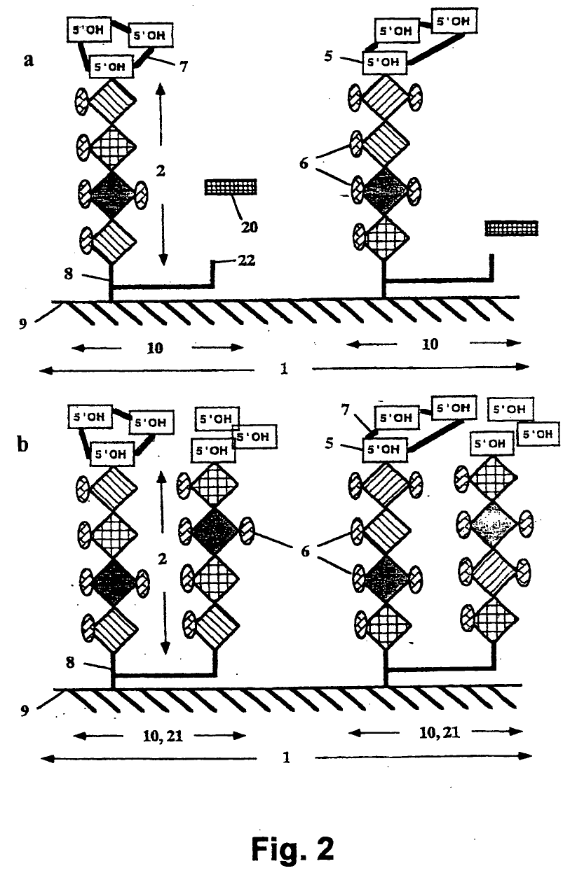

FIG. 2a diagrammatically illustrates the splitting of a second non-permanent protecting group 20 different to the first temporary protecting group 4 on the support 9, giving rise to a reactive group 22 on the support,

FIG. 2b diagrammatically illustrates the structure of a second array of locally defined oligonucleotides 2 on the reactive group 22 by means of combinatorial synthesis,

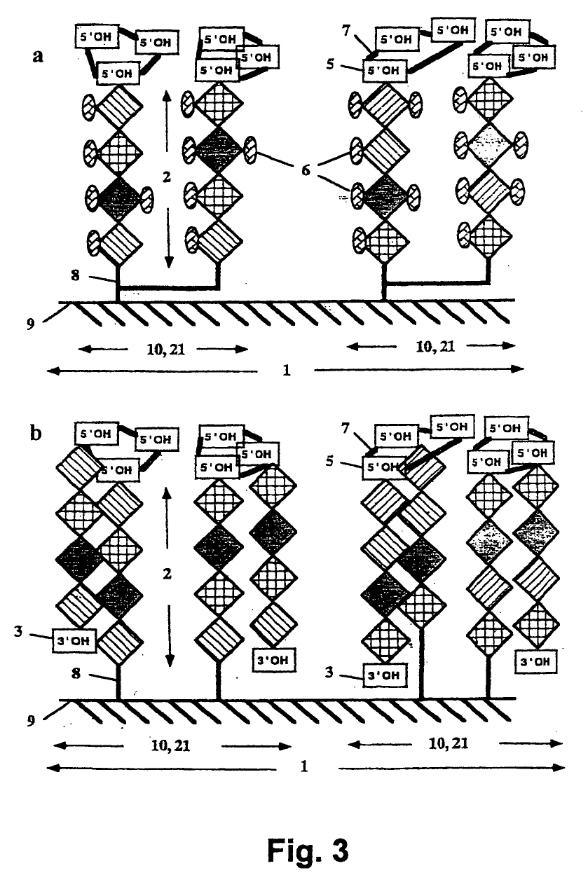

FIG. 3a diagrammatically illustrates the development of cross-linking 7 between the free 5′-OH ends 5 of the second array of locally defined oligonucleotides 2, producing an array of primer pairs 21 of defined sequence, which are defined locally in each case,

FIG. 3b diagrammatically illustrates splitting of the permanent protecting groups 6, whereby the choice of suitable “handles” 8 produces the majority of oligonucleotides 2 with a free 3′-OH end 3,

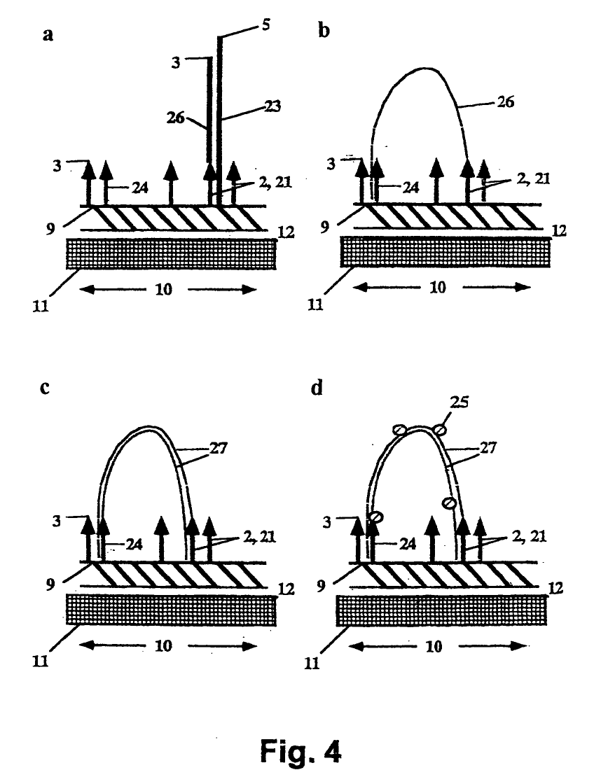

FIG. 4a diagrammatically illustrates the hybridisation of template DNA 23 at precisely defined locations 10 on a complementary primer 2, whereby DNA polymerase based on the 3′OH end 3 of the primers 2 synthesises the counter-strand 26 and a controllable heating block 11 supplies the hybridisation temperature, the temperature for the DNA polymerisation and the temperature for melting of the DNA double strand,

FIG. 4b diagrammatically illustrates the hybridisation of the DNA single strand 26 synthesised onto the 3′OH end 3 of the primer 2 following melting of the DNA double strand onto the complementary counter-strand primer 24,

FIG. 4c diagrammatically illustrates the formation of a double-strand PCR product 27 following repeated DNA polymerisation,

FIG. 4d diagrammatically illustrates the incorporation or intercalation of a fluorescent dye 25 serving as evidence of the PCR product 27 into the resulting locally defined PCR product 27, which can be increased by additional PCR cycles,

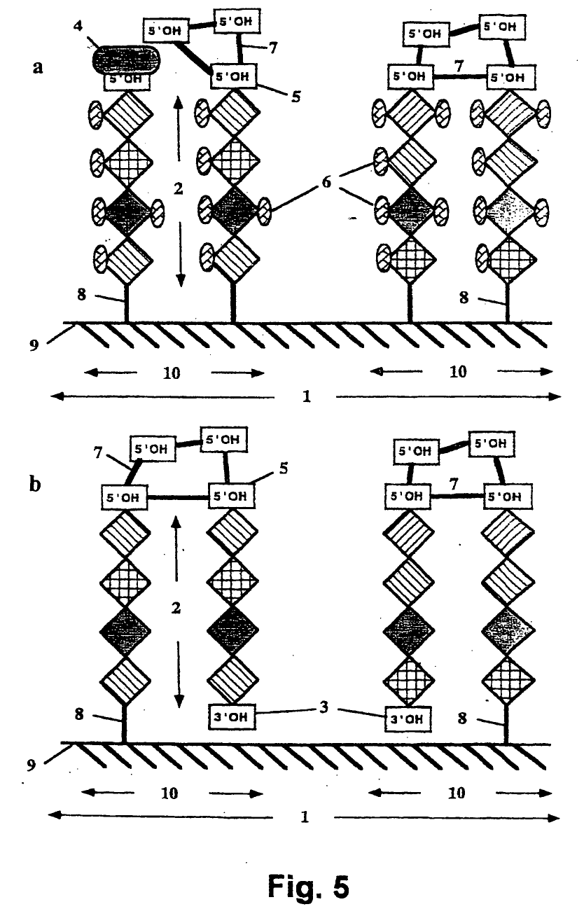

FIG. 5a+5b diagrammatically illustrates a method for synthesis of a support-bound array 1 of oligonucleotides 2 with free 3′OH ends 3, whereby on completion of combinatorial synthesis (FIG. 5, I.) the temporary protecting group 4 is split from the 5′OH end 5 and then, before splitting of the permanent protecting groups 6, the free 5′OH ends 5 are cross-linked, at which point (FIG. 5, II.) the covalent bond 8 is removed via the 3′OH end of the synthesised oligonucleotides to the support 9 by a portion, preferably an overwhelming portion of the synthesised oligonucleotides or oligoribonucleotides 2, resulting in free 3′OH ends 3 and, after splitting of these 3′OH ends 3 from the support 9 a portion, preferably a predominant portion of the synthesised oligonucleotides or oligoribonucleotides 2 can bind covalently to the support 9 due to the cross-linking 7 at the 5′OH end 5,

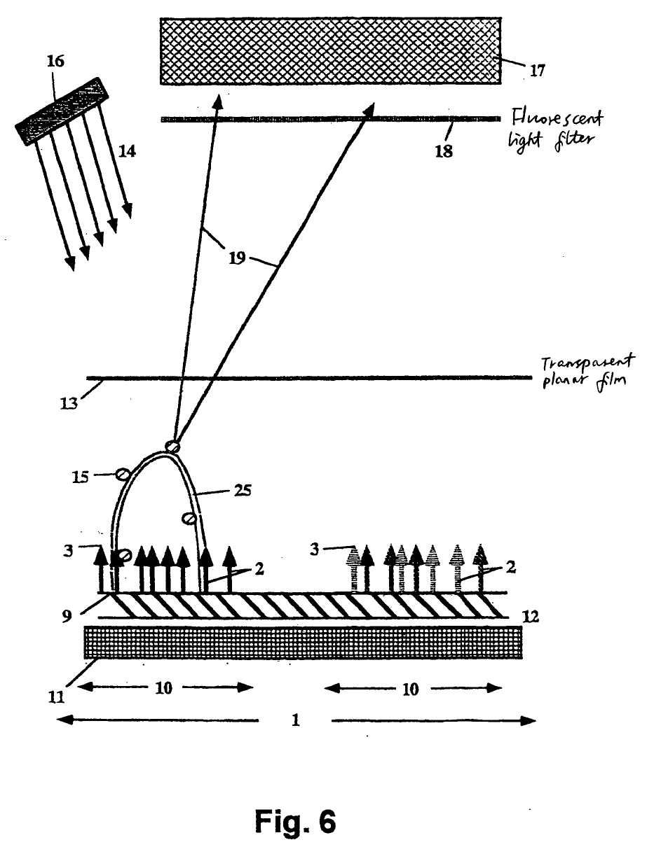

FIG. 6 diagrammatically illustrates a device for parallel analysis of PCR reactions, in which the metallic heating block 11 is milled planar and a fluorescent light filter 18, which collimates excitation light 14, but admits emitted fluorescent light 19, is provided between a detection unit 17 and an array 1 of oligonucleotides 2,

FIG. 7a diagrammatically illustrates the synthesis of a oligonucleotide 2 bound on a support 9 at 8 and the splitting of a first temporary protecting group 4. The result is a free 5′OH-group. The synthesis artefact 28 resulting from synthesis and bound to the support 9 was provided during synthesis with a “cap” 29,

FIG. 7b diagrammatically illustrates the development of cross-linking 7 between the free 5′OH ends 5. Due to the “caps” 29 on the 5′OH end 5 the synthesis artefact 28 is not cross-linked at the same time,

FIG. 7c diagrammatically illustrates the splitting of the permanent protecting groups 6 and the (partial) splitting of the connection of the oligonucleotide 2 to the support 9, whereby the choice of suitable “handles” 8 produces the multiplicity of oligonucleotides 2 with free 3′OH end 3. Because the synthesis artefact 28 is no longer covalently attached to the support 9 after this, it can be washed away, which leads to (partial) in situ cleaning of the oligonucleotide 2.

For the sake of clarity only oligonucleotides (2) are mentioned in the description of FIG. 1 to FIG. 7. But the description applies as well for other oligomers (2), as also evident from the labelling table. There are many combinatorial syntheses, where each of the different monomers have two distinguishable ends A (3) and B (5). In nucleotides these are the 3′OH end and the 5′OH end, in amino acids the COOH group and the NH₂ group, and in other monomers these are other groups again. The invention relates to all these different combinatorial syntheses.

It should be pointed out here that the invention enables not only the synthesis of oligonucleotides (2) with a free 3′OH end (3), but also enables in situ cleaning of support-bound oligomers (2). The longer an oligomer becomes during combinatorial synthesis, the more support-bound synthesis artefacts 28 are produced (illustrated in FIG. 7), which can make up by far the major portion of the synthesised oligomers. This interferes in very many applications, since it is preferred to ascribe a bond or another observable effect to the defined oligomer, whose sequence is known, and not to an undefined artefact. For the most part these synthesis artefacts 28 result from the fact that after the temporary protecting groups (4) are split off not all reactive groups react with a new monomer. These are normally “capped” 29 before a new coupling cycle commences with splitting of the temporary protecting group (4). But “capping” 29 causes the synthesis artefacts 28 to lose their reactive group, to which they can also not be cross-linked (7). If a portion (preferably a very substantial portion) of the ends A (3) is split off from the support following cross-linking (7) (FIG. 3b), the equivalent portion of synthesis artefacts 28 is split off. Because these are not cross-linked (7), they no longer bind to the support (9) and can be washed away.

To achieve synthesis of defined primer pairs 21 fixed to a support with free 3′-OH ends each at a defined location, the combinatorial syntheses known from the prior art are altered as follows (see FIG. 1, FIG. 2 and FIG. 3).

As indicated diagrammatically in FIGS. 1a and 1b, following combinatorial synthesis of a first array 1 of oligonucleotides 2 bound at specific, precisely defined locations 10 on the support 9, in a first step the temporary protecting groups 4 (an example for such a protecting group is 4′,4′-dimethoxytritylchloride (DMTr)) are split off at the 5′-OH end 5 of the oligonucleotides 2 and then the now free 5′-OH ends 5 are cross-linked via cross-linking 7. This is done in a manner known per se.

As illustrated diagrammatically in FIG. 1b and FIG. 2a, a second non-permanent protecting group 20 (e.g. 2-acetyl-5, 5-dimethyl-1, 3-cyclohexandione, (Dde)) different to the abovementioned first temporary protecting group 4 is then split on the support 9 in a second step. This occurs likewise in a manner known per se. The second non-permanent protecting group 20 can be introduced during the abovementioned cross-linking of the free 5′-OH ends or can have been applied before synthesis of the first array 1 of oligonucleotides 2 bound at specific locations 10 to the support 9.

In a third step, illustrated diagrammatically in FIGS. 2a and 2b and FIG. 3a, splitting of the second non-permanent protecting group 20 on the support 9 produces a reactive group 22 (e.g. a hydroxyl group or an amino group), on which, by means of known combinatorial syntheses, a second array of locally defined oligonucleotides 2 can be built up, whose free 5′-OH ends 5 are cross-linked as described hereinabove (cross-linking 7). This results in an array of primer pairs 21 of defined sequence, which are each locally defined.

Next, as shown in FIG. 3b, in a fourth step the permanent protecting groups 6 are split off in a manner known per se, whereby the choice of suitable “handles” 8 results in the majority of the oligonucleotides 2 now having free 3′-OH ends 3. Further anchoring 8 of the oligonucleotides 2 is preferably achieved via incomplete splitting of the abovementioned handles 8, e.g. by deriving of the support 9 with a mixture of handles 8, of which one portion is split under selected conditions, while a preferably smaller portion of the handles 8 remains covalently attached to the support 9 under the selected conditions.

Some of the advantages of these methods according to the present invention are that:

an array of defined oligonucleotide pairs results instead of an array of individual locally defined oligonucleotides,

the oligonucleotides have free 3′-OH ends instead of a free 5′-OH end and thus constitute a template-dependent potential substrate for DNA polymerases,

in situ cleaning of the defined oligonucleotides takes place.

In particular the cleaning of the oligonucleotides mentioned above only by way of a non-limiting example has major advantages in practice and can be put to use without problem with other oligomers also, such as e.g. RNA or peptides, instead of with oligonucleotides. In previously known methods the majority of synthesis artefacts 28 has no free 5′-OH end and is accordingly not cross-linked in the abovedescribed first step at 7. In the abovedescribed fourth step, however, the majority of the synthesis artefacts 28 is split off together with the oligonucleotides cross-linked at the 5′OH end also and can then be washed away from the support.

The abovedescribed primer pairs 21 can act as locally defined “solid-phase PCR”. Compared to conventional PCR methods thousands of PCR reactions can be carried out in one reaction vessel very easily. FIG. 4a shows how template DNA 23 hybridises to a complementary primer 2 at precisely defined locations 10. A DNA polymerase synthesises the counter-strand 26 based on the 3′OH end 3 of the primer 2. A controllable heating block 11 supplies the heat required for hybridisation, for DNA polymerisation and for melting of the DNA double strand. To avoid steric problems, which can occur via the double-helix structure of DNA, a preferably thermostable enzymatic activity (e.g. a helicase, a gyrase or topoisomerase together with ATP), which unravels superhelical twists, can be added to the solid phase PCR. This applies especially to solid-phase PCRs, in which longer DNA zones are duplicated.

FIG. 4b shows how, after melting of the DNA double strand, the DNA single strand 26 synthesised onto the 3′OH end 3 of the primer 2, as shown in FIG. 4a, hybridises on the complementary counter-strand primer 24.

FIG. 4c illustrates the formation of a double-strand PCR product 27 after renewed DNA polymerisation.

A fluorescent dye 25 serving as proof of the resulting locally defined PCR product 27, which can be increased by additional PCR cycles, can be incorporated or intercalated as indicated in FIG. 4d.

With reference to FIG. 5 a method for synthesis of a support-bound array 1 of oligonucleotides 2 with free 3′OH ends 3 will be described hereinbelow.

As shown in FIG. 5a, on completion of the combinatorial synthesis the temporary protecting group 4 is removed from the 5′OH end 5. Next the free 5′OH ends 5 are attached via cross-linking 7 prior to splitting of the permanent protecting groups 6.

Then, as shown in FIG. 5b, the covalent bond 8 is removed via the 3′OH end of the synthesised oligonucleotides onto the support 9 by a portion, preferably a predominant portion of the synthesised oligonucleotides or oligoribonucleotides 2, producing free 3′OH ends 3. Following splitting of these 3′OH ends 3 from the support 9 a portion, preferably a predominant portion, of the synthesised oligonucleotides or oligoribonucleotides 2 binds covalently 8 to the support 9 due to the abovementioned cross-linking 7 at the 5′OH end 5.

FIG. 6 diagrammatically illustrates a device for parallel analysis of PCR reactions, in which the metallic heating block 11 present in most commercially available PCR machines was milled planar, enabling close contact 12 with a planar array 1 of oligonucleotides 2 with free 3′OH ends 3. With use of such a device an array 1 of oligonucleotides 2 is covered with an interchangeable, transparent planar film or plate 13, especially also transparent for UV light, which can be fixed on the array 1, to prevent evaporation of the reaction buffer.

To enable parallel proof of the double-strand DNA 25 resulting from the PCR reactions, the abovementioned array 1 of oligonucleotides 2 is irradiated with excitation light 14 which is particularly suited to excite the fluorescent dye 15 associated with the formed double-strand DNA 25. The fluorescent dye 15 is excited by an excitation light source 16 mounted over the array 1 of oligonucleotides 2.

Mounted between the detection unit 17, which particularly advantageously comprises a digital camera, and the abovementioned array 1 of oligonucleotides 2 is a fluorescent light filter 18 which collimates the excitation light 14, but allows the emitted fluorescent light 19 to pass through.

The data recorded by the detection unit 17 are transferred to an essentially commercial computer where they undergo image analysis. The abovedescribed device for parallel analysis of PCR reactions is especially suited to parallelised “online” detection of PCR reactions.

FIG. 7 schematically depicts the in situ cleaning of oligomers 2 bound on the support 9 at 8. The synthesis artefact 28 resulting from synthesis and bound on the support 9 at 8 was provided during synthesis with a “cap” 29, so that this molecule 28 cannot take part in the development of cross-linking 7 between the free ends 5. After the permanent protecting groups 6 are removed and after (partial) splitting of the connection 8 of the oligomer 2 to the support 9 the synthesis artefact 28 can be washed away, resulting in (partial) in situ cleaning of the oligomer 2. The percentage of the oligomer 2 bound on the support 9 at 8 and thus also the degree of cleaning is determined by the choice of “handles” 8. It should be mentioned here that this in situ cleaning method is suitable for all combinatorial syntheses.

A device according to the present invention for parallel analysis of PCR reactions will be described hereinbelow by means of an array of oligonucleotides with free 3′OH ends, as will the use of such a device.

The device for analysis of PCR reactions is based substantially on commercial PCR machines, which are modified as described hereinafter, to enable particularly advantageous analysis, which is very simple, of many PCR reactions at the same time:

a) The metallic heating block present in most commercial PCR machines is milled planar, enabling close contact with a planar array of oligonucleotides with free 3′OH ends.

b) The abovementioned array of oligonucleotides is covered with an interchangeable, transparent, in particular also transparent for UV light, planar film or plate, which can be fixed on the array, thus avoiding evaporation of the reaction buffer.

c) For parallel analysis of PCR reactions the abovementioned array of oligonucleotides is irradiated with excitation light, with UV light especially, which is particularly suited for exciting a fluorescent dye, in particular ethidium bromide.

d) The abovementioned excitation of the fluorescent dye occurs via an excitation light source mounted over the abovementioned array of oligonucleotides.

e) Mounted between the detection unit, which particularly advantageously comprises a digital camera, and the abovementioned array of oligonucleotides is a fluorescent light filter which collimates the excitation light, but allows the emitted fluorescent light to pass through.

f) The data recorded by the detection unit are transferred to an essentially commercial computer where they undergo image analysis.

Arrays of oligonucleotides, produced according to the present invention, with free 3′OH ends for parallel analysis of PCR reactions and their possible applications will be described hereinbelow.

Materials produced according to the present invention contain the molecule libraries produced using the abovementioned methods, materials or devices, in particular oligonucleotide libraries with free 3′OH-ends. An indicator of these oligonucleotide libraries is that,

they result from combinatorial synthesis of a limited number of suitable nucleoside monomers,

they are present as a 2-dimensional array on a suitable derived support, whereby the individual components of the oligonucleotide library can be assigned locally (the support is derived in a manner known per se),

they have a free 3′OH end, which, following hybridisation of template DNA, is a substrate of template-dependent DNA polymerase,

preferably two defined oligonucleotides were synthesised per defined location.

The abovementioned oligonucleotide libraries serve as very simple PCR analysis, in particular of complex template mixtures.

With one type of array the primer pairs of appropriate sequence synthesised thereon duplicate areas of preferably a plurality of different genes of pathogens, so that at the same time a diagnosis can be made as to whether there is an infection with one of these pathogens or not. To prevent steric problems, which may arise from the double-helix structure of DNA, a preferably thermostable enzymatic activity (e.g. a helicase, a gyrase or topoisomerase together with ATP), which unravels superhelical twists, is added to the solid-phase PCR.

For example, 1000 infectious diseases each with 100 different primer pairs can be covered on an array with 10⁵ different primer pairs, including almost all known human-pathogenic virus genomes (e.g. Hepatitis A, B, C, HIV, Papilloma viruses, Rhino viruses, Influenza viruses etc.), bacterial—genomes (e.g. Helicobacter pylori, Haemophilus influenza, E.coli etc.) and the genomes of various human-pathogenic single cells (e.g. Entamoebae histolitica, Plasmodium, Trypanosomen etc.).

In another type of array the primer pairs of suitable sequence synthesised thereon duplicate zones of preferably the most possible human ESTs (Expressed Sequence Tags), so that so-called “Expression Profiling” can be performed after hybridisation of complex cDNA. At the same time parallel analysis is performed as to which and how many mRNAs (and thus the cDNAs derived therefrom) are expressed in a preferably human tissue or a preferably human cell line. In particular this type of array is suited to the comparison of several complex cDNAs to one another and thus to identifying comparatively upwards or downwards adjusted genes.

Genomic DNA can also be analysed using this type of array. In the process a quantitative PCR e.g. can be used to find out which genome areas are deleted homozygously or heterozygously, with the aim of assigning these areas again to a genetic disease.

In another application this type of array acts as proof of polymorphisms and thus e.g. for fine-mapping disease genes. A computer program scans the sequences of the human chromosome 22 available in the databanks for restriction sites which are separated from one another by 100 to 300 Bp. The same program then constructs primer pairs, which each contain one of the abovementioned two restriction sites already in the primer sequence, which in each case contain other restriction sites only in the DNA duplicated by the primer pair. With many of these primer pairs an array of primer pairs is produced using the methods described here, whereby the individual primer pairs are preferably present in a linear arrangement corresponding to their position on human chromosome 22.

High-grade parallelised PCR is carried out using such an array, as already described, typically with comparison of results, which are obtained with the genomic DNA of the father, mother and child as template.

If a genetic polymorphism is located in the region of each second restriction site, then it can very easily be proven by digestion with the corresponding restriction enzyme. If both chromosomes each code both restriction sites, the result with restrictive digestion is a diffuse halo locally defined by the position of the primer pairs. This halo results from the diffusing PCR amplificate, dyed with ethidium bromide for example. If only one chromosome codes each of the two restriction sites, then a sharply delimited core of the support-bound PCR amplificate remains within the comparatively weaker diffuse halo. If the abovementioned second restriction site is missing on both chromosomes, then the abovementioned halo is missing after restrictive digestion. If the images of father, mother and child are now superposed graphically (e.g. by allocation of false colours), then the chromosomal areas of the child can be assigned very easily and at the same time very precisely to either the father or the mother.

The abovementioned arrays of primer pairs can be reused if the filters can be digested with suitable restriction endonucleases on completion of PCR reaction, and then heated and non-support-bound DNA is then washed away. The prerequisite is that the 3′ ends of the used primers contain one of several suitable recognition sequences for the abovementioned restriction endonucleases. This method is especially useful if two different complex templates are compared to one another, whereby comparing quantitative data should be obtained and variations in the filter production are to be avoided as far as possible.

A special characteristic of the abovementioned arrays is a previously unattained analysis sensitivity, since the measuring principle of these arrays is based on polymerase chain reaction (PCR), which when compared to hybridisation employed to date is substantially more sensitive.

The abovedescribed method enables production of an array of oligonucleotides, each having free 3′OH-ends. If template DNA is hybridised onto such an array of oligonucleotides, then the resulting complex comprising support-bound oligonucleotide and template DNA can serve as a substrate for DNA-dependent DNA polymerases. In the case of an RNA template an RNA-dependent DNA polymerase can be used in its place. Such arrays can be utilised for instance for parallel sequencing of complex templates and/or for high-grade parallelised polymerase chain reactions. The specialist is well aware of the required reaction conditions.

In the case of analysis of high-grade parallelised polymerase chain reactions it is especially advantageous if, as described hereinabove, instead of a defined oligonucleotide two defined oligonucleotides are synthesised according to location. Here in particular for subsequent parallel sequencing of complex templates each one of the primers of the primer pairs can be presented in a clearly smaller quantity, compared to the other primer, such that predominantly single-strand DNA is produced after a few multiplication cycles. If ddNTPs are added in at this stage the result is a family of single-strand oligonucleotides, which e.g. enables determination of sequence of the multiplied template DNA through its analysis in a mass spectrometer.

The high specificity typical for PCR can be achieved very easily by means of a primer pair, while a single primer at least with a complex mixture of templates would yield a large portion of artefacts.

Another important point is that in the case of the abovementioned arrays the primers are fixed on the support, since this enables parallel analysis of many PCR reactions simultaneously in one reaction vessel, because the otherwise resulting primer mixture would lead to unforeseeable amplificates. This design even allows the template DNA being used to be washed away after one or several PCR cycles, so that the newly resulting template DNA is also present only in support-bound form. This further reduces the proportion of PCR artefacts.

The high-grade parallelised polymerase chain reactions are analysed effectively using the device according to the present invention for parallel analysis of PCR reactions, described in greater detail hereinabove.

The individual fluorescising points, which are allocated to the corresponding primer pairs defined according to location, can be analysed on completion of the PCR reaction or, in an especially advantageous way, “online” with the abovementioned inventive device for parallel analysis of PCR reactions. Also, the individual fluorescising points are assigned to the corresponding primer pairs defined by location, though this method for each primer pair defined by location gives a flow curve of the PCR reaction, so that the PCR reaction of each primer pair can be quantified.

The fact that free nucleotides or single-strand DNA such as the abovementioned oligonucleotide primer give off a considerably weaker fluorescent signal with intercalated ethidium bromide than double-strand DNA can be utilised to particular advantage.

There are also more methods known to the expert for quantifying PCR reactions, which likewise are based on incorporating or intercalating fluorochromes into the double-strand DNA formed during PCR reaction. Not least there is also the option of directly pursuing the change in absorption, occurring with conversion of mononucleotides into double-strand DNA.

The abovementioned arrays of primer pairs can be reused if the filters can be digested with suitable restriction endonucleases on completion of PCR reaction, and non-support-bound DNA is then washed away. The prerequisite is that the 3′OH ends of the used primers contain one of several suitable recognition sequences for the abovementioned restriction endonucleases. This method is especially useful if two different complex templates are compared to one another, whereby comparing quantitative data should be obtained and variations in the filter production are to be avoided as far as possible.

A particularly advantageous use of the abovementioned arrays results from almost completely automated parallel diagnosis of very many different diseases, in particular infectious diseases, by means of PCR. In the process the diagnosis safety for the individual diseases can also be considerably increased, in that not only one, but many disease-specific primer pairs are analysed.

Another particularly advantageous use of the abovementioned arrays is preparing an expression pattern, in particular a comparable expression pattern. The mRNA of a tissue or a cell line is transcribed into (high-complex) cDNA in a manner known per se, which again serves as template for the abovementioned arrays. If these arrays now carry primer pairs, which are capable of multiplying human EST sequences (Expressed Sequence Tag), then the EST sequences present in the abovementioned mRNA or cDNA are increased as defined by location and multiplied almost quantitatively. In particular comparison of tumorous tissue to the surrounding normal tissue results in EST sequences which are expressed comparatively strongly or weakly in the tumorous tissue. Due to the considerable sensitivity of PCR technology weakly expressed genes are also available for analysis and comparatively minimal quantities of templates can be used, where these filters are far superior to the prior art.

Yet another especially advantageous application of the abovementioned array is the assigning of homozygously or heterozygously deleted areas to somatic genetic diseases and hereditary diseases. This can be discovered by means of the quantitative PCR described in greater detail hereinabove. For this, an array with suitable primer pairs has to be used which duplicate sequential areas of those genes which code the ESTs mentioned hereinabove.

A number of examples for carrying into effect methods according to the present invention or the use of devices according to the present invention is now described:

A) Synthesis of an array of primer pairs with each free 3′OH end on a support by means of a modified colour laser printer.

A suitable support with free amino groups (or hydroxyl groups) is manufactured using standard methods. If not already available via the first step, an appropriate linker is synthesised onto the free amino groups (or hydroxyl groups) by means of standard synthesis familiar to the expert under water-free conditions.

This linker preferably comprises Dde-Fmoc-Lys, whose one amino group is blocked by a fmoc-protecting group, the other being blocked by a Dde-protecting group. The fmoc-protecting group is split off at 25° C. for 10 minutes with 20% piperidine in DMF, i.e. under conditions in which the Dde-protecting group remains stable. Next, techniques known to the expert are used to activate one or two RNA phosphoramidites with the aid of tetrazole and attach them to the support. A small portion of less than 5% of the corresponding DNA phosphoramidites can be added during the coupling reaction.

After removal of the DMTr-protecting group from the 5′OH end of the ribonucleoside monomer attached to the support, the support is printed with 4 different toners containing the 4 different phosphoramidite monomers. The phosphoramidites are activated using tetrazole, attached to the support, unattached monomers are washed away and the DMTr-protecting group is then removed from the 5′OH end of the growing oligonucleotide. Repetition of this procedure leads to combinatorial synthesis of oligonucleotides.

Coupling of the activated phosphoramidites (with protecting groups) to the support, splitting the protecting groups and the washing steps take place under standard conditions for oligonucleotide synthesis known to the expert.

After such a first array of oligonucleotides was synthesised, at the end the DMTr protecting group is removed from the 5′OH end and the free 5′OH groups are cross-linked e.g. with cross-linked cellulose acetate, or EDTA, or triethylenetetramine hexaacetic acid and N,N′-dicyclohexylcarbodiimide (DCC) under conditions familiar to the expert. The previous introduction of an amino group at the 5′OH end is especially suited to this. This can be achieved e.g. with the 5′ Amino Modifier 5 (Eurogentec; #10190502).

The abovementioned Dde protecting group is then removed at 25° C. with 2% hydrazine in DMF for 10 minutes and one or two RNA phosphoramidites are first synthesised to the amino acid of the abovementioned lysine linker, as described hereinabove. At the same time the abovementioned RNA phosphoramidites can be printed out as phosphoramidite toner particles or distributed evenly over the support in coupling buffer. As explained above, during the coupling reaction a small portion of the corresponding DNA phosphoramidites can be mixed in during the coupling reaction.

An array of oligonucleotides is again synthesised and cross-linked at the 5′OH end, as described hereinabove. Finally all protecting groups are removed with ammonia for 45 minutes at 55° C. to 70° C., and the support is washed with acetonitrile and dried, resulting in a support with various defined areas which represent a pair of oligonucleotides in each case. In the given example the pairs of oligonucleotides represent sequences for duplicating ca. 50,000 different human ESTs. In a final step the 3′OH ends of the oligonucleotides are removed from the support with RNase or split under alkaline conditions.

B) Comparison of the gene expression profiles of tumorous tissue with normal tissue

mRNA is obtained from the tumorous tissue of a patient and from the surrounding normal tissue using techniques known to the expert; this is transcribed in cDNA and used as complex template for hybridisation on the arrays described under A).

The abovementioned array, together with the abovementioned template cDNA is clamped into the abovedescribed device for parallel analysis of PCR reactions. Then known suitable thermophilic DNA polymerases in an appropriate buffer are added and the PCR reaction is started. By using the device of FIG. 6 above a flow curve of the PCR reaction is prepared for each defined primer pair, allowing a value characterising this flow curve to be assigned to each of the ESTs defined by the primer pairs.

The values obtained with cDNA from the tumorous tissue of a patient and the values obtained with cDNA from the surrounding normal tissue are compared to one another. This leads to identification of genes which are underexpressed or overexpressed in the tumorous tissue compared to the normal tissue.

A further application example is parallel PCR diagnostics of pathogens.

1. A method for synthesis of a support-bound array (1) of oligomers, in particular oligonucleotides (2) or oligoribonucleotides, with free ends A, in particular with free 3′OH ends (3), whereby a temporary protecting group (4) is provided on end B, preferably on the 5′OH end (5) of the oligomer, and permanent protecting groups (6) are provided on reactive side groups,
characterised,
in that the temporary protecting group (4) is removed from the end B, in particular from the 5′OH end (5), on completion of the combinatorial synthesis of the oligomers (2) on the support (9),
in that the free ends B, in particular the 5′OH ends (5), are then cross-linked via cross-linking (7) even before the permanent protecting groups (6) are removed,
in that the covalent bond (8) of the synthesised oligomers (2), in particular of the oligonucleotides (2) or oligoribonucleotides (2) can then be split off via the end A, in particular the 3′OH end, onto the support (9) by a portion, preferably by a predominant portion of the synthesised oligomer (2), resulting in free ends A, in particular free 3′OH ends (3),
in that also a portion, preferably a predominant portion of the synthesised oligomers (2), binds covalently (8) to the support (9), due to the abovementioned cross-linking (7) at the end B, in particular at the 5′OH end (5), after removal of the ends A, in particular the 3′OH ends (3), from the support (9).

2. The method for synthesis of a support-bound array (1) of oligomers, in particular oligonucleotides (2) or oligoribonucleotides, with free A ends, in particular with free 3′OH ends (3), whereby a first temporary protecting group (4) is provided at the end B, in particular at the 5′OH end (5) of the oligomers and permanent protecting groups (6) are provided on reactive side groups,
characterised,
in that in a first step following combinatorial synthesis of a first array (1) of oligomers (2) bound at specific, precisely defined locations (10) on the support (9) the first temporary protecting group (4) is removed at the end B, in particular at the 5′-OH end (5) of the oligomers, and the now free ends B are then either cross-linked via cross-linking (7) or are blocked by a second permanent protecting group (30),
in that in a second step a second temporary protecting group (20) different to the abovementioned first temporary protecting group 4) is removed on the support (9), resulting in a reactive group (22) on the support (9),
in that in a third step a second array of precisely defined oligomers (2) is constructed on the reactive group (22) preferably by means of combinatorial synthesis, whose free ends B, in particular whose free 5′-OH ends (5) can be cross-linked, as in the first step,
in that in a fourth step the permanent protecting groups (6, 30) are split off, whereby the choice of suitable handles (8) determines that the majority of oligomers are present with a free end A, in particular with free 3′-OH end (3).

3. The method as claimed in claim 1 or 2, characterised in that further anchoring of the oligomers by incomplete removal of the handles (8), in particular by derivation of the support (9) with a mixture of handles (8), whose one part is removed under the selected conditions, while a preferably smaller portion of the handles (8) remains covalently attached to the support (9) under the selected conditions.

4. The method as claimed in claim 2 or 3, characterised in that the second temporary protecting group (20) is introduced during cross-linking of the free 5′-OH ends in the first step or even before synthesis of the first array (1) of oligomers (2) bound at specific locations (10) to the support (9) has been applied.

5. A method for synthesis of a support-bound array (1) of oligomers, in particular oligonucleotides or oligoribonucleotides, with free 3′OH ends (3), whereby a temporary protecting group (4) is provided at the 5′OH end (5) of the oligomers and permanent protecting groups (6) are provided on reactive side groups,
characterised,
in that the temporary protecting group (4) is removed from the 5′OH end (5) on completion of combinatorial synthesis,
in that the free 5′OH ends (5) are then attached via cross-linking (7) even before the permanent protecting groups (6) are removed,
in that the covalent bond (8) is removed via the 3′OH end of the synthesised oligomers onto the support (9) by a portion, preferably by a predominant portion of the synthesised oligomers, resulting in free 3′OH ends (3), so that after the 3′OH ends are removed from the support a portion, preferably a predominant portion of the synthesised oligomers, can bind covalently onto the support (9) due to cross-linking (7) at the 5′OH end (5).

6. The method as claimed in any one of claims 1 to 5, characterised in that two different oligomers, in particular two different oligonucleotides or oligoribonucleotides are synthesised per defined location (10).

7. The method as claimed in any one of claims 1 to 6, characterised in that in the array (1) more than 100 oligomers defined by location per cm² are bound onto the support (9).

8. The method as claimed in any one of claims 1 to 7, characterised in that DNA, RNA or PNA is hybridised on the array (1).

9. The method as claimed in any one of claims 1 to 8, characterised in that the abovementioned arrays (1) are brought into contact with DNA polymerases or RNA polymerases.

10. The method as claimed in any one of claims 1 to 9, characterised in that the abovementioned arrays (1) are used for PCR analysis.

11. Solid phase PCR, whereby template DNA is hybridised at precisely defined locations (10) onto a primer synthesised on a support, as claimed in any one of claims 1 to 10, and the heat required in each case for hybridisation, for DNA polymerisation and for melting the DNA double strand is supplied by means of a controllable heating blocks (11).

12. A device for analysis of arrays (1) of oligomers,
characterised,
in that a heating block (11) is provided with a substantially planar contact surface,
in that means are provided for irradiating excitation light (14), especially UV light, onto an array positioned on the heating block,
in that a detection unit (17) is provided, in particular a CCD array for fluorescent light (19) emitted from the array of oligomers,
in that provided between the detection unit (17) and the array (1) to be examined is a fluorescent light filter (18) which collimates the excitation light (14), but admits the emitted fluorescent light (19), and
in that the abovementioned analysis is repeated, in particular as a time flow curve during an ongoing enzymatic reaction, in particular during a PCR reaction, which alters the abovementioned array of oligomers.

13. The method for analysis of the array (1) of oligomers synthesised as claimed in claim 1 to 10, using a device as claimed in claim 12,
characterised,
in that an array to be examined is placed into close contact with the heating block (11),
in that the array (1) is covered with a translucent planar film or plate (13) to avoid evaporation of a reaction buffer,
in that the array (1) for parallel analysis of PCR reactions is irradiated with excitation light (14), in particular with UV light,
in that the data recorded by the detection unit (17) are transferred to a commercial computer, where they undergo image analysis.

14. The method for analysis of the arrays (1) of oligomers synthesised as claimed in claim 1 to 10, using a device as claimed in claim 12, characterised in that the arrays (1) are analysed using the device mentioned in claim 12 above.

15. A support having at least one array of oligomers with free 3′OH ends for parallel analysis of PCR reactions, characterised in that the array was applied to the support as claimed in any one of the methods described in claims 1 to 10.

16. The support as claimed in claim 15 with an oligonucleotide library, characterised in that the oligonucleotide library has been created by combinatorial synthesis of a limited number of suitable monomers.

17. The support as claimed in claim 15 or 16 with an oligonucleotide library, characterised in that the oligonucleotide library is present as a 2-dimensional array on the support derived appropriately, whereby the individual components are assigned to the oligonucleotide library as defined by location.

18. The support as claimed in any one of claims 15 to 17 with an oligonucleotide library, characterised in that the oligonucleotide library has a free 3′OH end, which is a substrate of template-dependent DNA polymerases or RNA polymerases after hybridisation of template DNA.

19. The support as claimed in any one of claims 15 to 18 with an oligonucleotide library, characterised in that two defined oligonucleotides per defined location were synthesised in the oligonucleotide library.

20. The support with at least one array of oligomers, which have undergone an in situ cleaning step, characterised in that the array was applied to the support according to a method described as claimed in claims 1 to 10.

21. The method for synthesis of a support-bound array (1) of oligomer pairs (21), especially of oligonucleotides (2) or oligoribonucleotides,
characterised,
in that at least two oligomers (2) different to one another are synthesised or applied per defined location on an array (10), that both oligomers (2) have free 3′-OH ends (3) or are derived at free 3′-OH ends (3), and that a high-grade parallelised polymerase chain reaction (PCR) is carried out with the array of oligomer pairs (21) and that during the PCR reaction a preferably thermostable enzymatic activity, in particular a helicase, a gyrase or topoisomerase, is added along with ATP which unravels superhelical twists.
